(19) United States
(12) Patent Application Publication
(10) Pub. No.: US 2022/0125974 A1
(43) Pub. Date: Apr. 28, 2022

(54) PROTECTIVE BARRIER ASSEMBLY AND METHOD

(71) Applicant: GREAT BARRIER SOLUTIONS INC., Maple (CA)

(72) Inventors: GEORGE ANTHONY CALABRESE, MAPLE (CA); MATTHEW DAVID CALABRESE, ORLANDO, FL (US)

(73) Assignee: GREAT BARRIER SOLUTIONS INC., MAPLE (CA)

(21) Appl. No.: 17/505,729

(22) Filed: Oct. 20, 2021

Related U.S. Application Data
(60) Provisional application No. 63/104,028, filed on Oct. 22, 2020.

(51) Int. Cl. A61L 2/232; B65H 37/00

(52) U.S. Cl. CPC A61L 2/232; B65H 2555/25; B65H 37/00

(57) ABSTRACT

A flexible barrier assembly includes a frame defining a window, feed and collection reels mounted on the frame, a flexible protective barrier suspended and held taut between the feed and collection reels and overlaying the window, and a motor operatively coupled to the collection reel to rotate the collection reel. Rotation of the collection reel by the motor causes advancement of the protective barrier from the feed reel to the collection reel.

### CROSS-REFERENCES TO RELATED APPLICATIONS

This application claims the benefit of U.S. Provisional Patent Application No. 63/104,028 filed on Oct. 22, 2020, which is incorporated herein by reference in its entirety.

### FIELD

This document relates to protection from pathogens. More specifically, this document relates to assemblies that provide a protective barrier that can reduce the risk of exposure to infectious agents such as bacteria and viruses, and related methods.

### BACKGROUND

WO 2016/125140 (Shimon) describes a device for automatically providing a disposable seating-surface cover for a toilet-seat. The device includes a first housing adapted to feed a disposable seating-surface cover roll installed therein, in which the roll is a long strip layer that has a plurality of similar apertures, each of which defines an individual seating-surface cover. The device also includes a second housing adapted to receive and collect the roll by connecting an initial edge of the roll to a rotational mechanism situated in the second housing that is adapted to pull the roll such that each individual disposable seating-surface cover follows a pre-set displacing movement, in which one individual disposable seating-surface cover is positioned in a state of use. When an individual seating-surface cover of the roll is in a state of use, it lies directly on a seating surface of a toilet-seat, such that the aperture of the individual seating-surface cover is parallel to an aperture of the toilet seat.

US 2019/0083315 (Sigismondo et al.) describes a mud visor formed by a substantially transparent sheet attached to a goggle lens. The mud visor may be attached to the goggle lens using a substantially transparent adhesive or adhesive tape, such that the mud visor may provide clear and improved field of view for the user. Further, the mud visor may be configured to cover and guide a top portion of a roll-off film as the roll-off film is conveyed across the goggle lens. A top portion of the mud visor may be inserted into the lens groove of a goggle frame along with the goggle lens when the goggle lens is attached to the goggle frame. The mud visor may stretch across the goggle lens and overlap with film canisters at both sides to provide a seamless coverage to prevent mud from entering behind the mud visor or the roll-off film.

### SUMMARY

The following summary is intended to introduce the reader to various aspects of the detailed description, but not to define or delimit any invention.

Flexible barrier assemblies are disclosed.

According to some aspects, a flexible barrier assembly includes a frame defining a window, feed and collection reels mounted on the frame, a flexible protective barrier suspended and held taut between the feed and collection reels and overlaying the window, and a motor operatively coupled to the collection reel to rotate the collection reel. Rotation of the collection reel by the motor causes advancement of the protective barrier from the feed reel to the collection reel.

According to some aspects, a flexible barrier assembly includes a frame defining a window, where the frame supports an axle for receiving a feed reel carrying a flexible protective barrier film and a driveshaft for receiving a collection reel. The axle and driveshaft are positioned on the frame to suspend the flexible protective barrier between the feed and collection reels and overlaying the window. A motor is operatively coupled to the driveshaft to rotate the driveshaft. When the feed reel is received on the axle and the collection reel is received on the driveshaft, rotation of the driveshaft causes rotation of collection reel to cause advancement of the protective barrier from the feed reel to the collection reel.

Computer terminals are also disclosed. According to some aspects, a computer terminal includes a touch-sensitive screen, feed and collection reels mounted on either side of the touch-sensitive screen, a flexible protective barrier that is suspended and held taut between the feed and collection reels and positioned to overlay the touch-sensitive screen, and a motor operatively coupled to the collection reel to rotate the collection reel. Rotation of the collection reel by the motor causes advancement of the protective barrier from the feed reel to the collection reel.

Methods for controlling a flexible protective barrier are also disclosed. According to some aspects, a method is disclosed for controlling a flexible protective barrier that is suspended and held taut between feed and collection reels and positioned overlaying a window. The method includes detecting an event; and in response to detection of the event, automatically advancing the flexible protective barrier across the window by driving a motor.

### BRIEF DESCRIPTION OF THE DRAWINGS

The drawings included herewith are for illustrating various examples of articles, methods, and apparatuses of the present specification and are not intended to limit the scope of what is described in any way. Throughout the present description reference will be made to the following drawings:

FIG. 1A is a front perspective view of an embodiment of a protective barrier assembly according to the description, with housings thereof partially removed to show the interior thereof;

FIG. 1B is a rear perspective view of the protective barrier assembly shown in FIG. 1A, with the housings thereof partially removed;

FIG. 1C is a front view of the protective barrier assembly shown in FIG. 1A with the housings thereof partially removed;

FIG. 2A is a side view of an embodiment of a drive shaft that may be included in the protective barrier assembly shown in FIGS. 1A-C;

FIG. 2B is a front view of the drive shaft shown in FIG. 2A;

FIG. 3A is a close-up of a top end of the drive shaft shown in FIGS. 2A-B when installed in the protective barrier assembly of FIGS. 1A-C;

FIG. 3B is a close-up of a bottom end of the drive shaft shown in FIGS. 2A-B when installed in the protective barrier assembly of FIGS. 1A-C;

FIG. 4 is front perspective view of the protective barrier assembly shown in FIGS. 1A-C mounted to a computer terminal that includes a touch-sensitive screen;

FIG. 5 is front perspective view of a computer terminal that includes a touch-sensitive screen and an integrated protective barrier assembly;

FIG. 6 is a flow diagram illustrating a method of controlling a protective barrier assembly installed on a computer terminal;

FIG. 7 is a perspective view of a flexible protective barrier that may be loaded into the protective barrier assembly of FIGS. 1A-C, including a close-up of an attachment mechanism for a collection reel;

FIG. 8 is a perspective view of the flexible protective barrier shown in FIG. 7, attached to a collection reel, also showing a close-up view of an end of the collection reel;

FIG. 9A is a perspective view of another embodiment of a protective barrier assembly according to the description;

FIG. 9B is a perspective view of a variation of the protective barrier assembly shown in FIG. 9A;

FIG. 10 is a perspective view of yet another embodiment of a protective barrier assembly according to the description;

FIG. 11A is a front perspective view of yet another embodiment of a protective barrier assembly according to the description; and

FIG. 11B is a front perspective view of the protective barrier assembly shown in FIG. 11A, with an external housing removed.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Computer terminals equipped with touch-sensitive screens (also referred to herein simply as “touch screens”), keypads, button arrays, and other types/configurations of user interfaces are increasingly being deployed in public spaces, such as (but not limited to) cash registers, credit card and self-checkout point-of-sale (POS) terminals at stores and supermarkets, bank machines, information screens in malls, airport kiosks, gaming screens in casinos, and many other public spaces. While there may be many advantages conferred by the use of touch-sensitive screens and other user interfaces, such as ease of operation and greater user functionality, one potential adverse consequence is an elevated risk of transmission of infectious agents (also referred to herein as “germs”), such as bacteria and viruses, from person to person through surface contact with the user interface. That is, without frequent cleaning or disinfecting of the touch screen, for example, germs deposited on the screen surface by one user can cause illness or other health-related issues in another person who picks up those germs on their hands in a subsequent use of the same touch screen.

Even without shared use of publicly accessible touch screens, many common situations encountered in daily life still carry a risk of airborne transmission of infectious agents. For example, face to face interactions at checkout counters, dining at restaurants, prolonged presence at office workstations, hospital or medical center waiting rooms, or visiting a library just to name a few, can all place people into close enough proximity with one another that they may be at elevated risk of transmitting droplets or airborne germs to one another.

Plexiglass barriers can in some cases prove effective at reducing the risk of spread. However, such permanent barriers are limited by the fact that without frequent cleaning, there is still the possibility of infectious agents surviving for extended periods of time on the surface and thereby still transmitting from person to person. Furthermore, because it will not transmit gestures, a rigid material like plexiglass also cannot effectively shield a touch-sensitive screen, keypad, or other user interface as described herein from surface contaminants without critically impairing their functionality.

Generally disclosed herein is an assembly (and related method) that can protect users from transmission of germs due to contact with touch screens and other user interfaces. The assembly can be installed on a publicly used computer terminal, either as a retro-fit to the computer terminal, or integrated with the computer terminal as an “all-in-one” unit. The assembly provides a flexible protective barrier that is initially provided on a reel (a “feed reel”). The flexible protective barrier is advanced over, e.g., a touch screen and collected on a collection reel, so that the flexible protective barrier is suspended and held taut between the feed reel and the collection reel, and is positioned between the user and the touch screen. The user thus interacts with the touch screen via the flexible protective barrier, so that any germs are transmitted to the flexible protective barrier, as opposed to the touch screen itself. When the user has finished using the computer terminal (e.g., upon completion of a transaction or upon walking away), the flexible protective barrier is advanced, so that the segment of the flexible protective barrier touched by or presented to the user, and thus potentially contaminated with germs, is collected on the collection reel, and a new segment of the flexible protective barrier is dispensed from the feed reel to be presented to the next user of the computer terminal. Thus, each user touches and interacts with a fresh segment of the flexible protective barrier, to protect each user from germs from a previous user.

In addition to use with computer terminals, the assemblies disclosed herein can be used to provide a barrier between individuals, such as at checkout counters, service windows, office workstations, waiting rooms, or in restaurants.

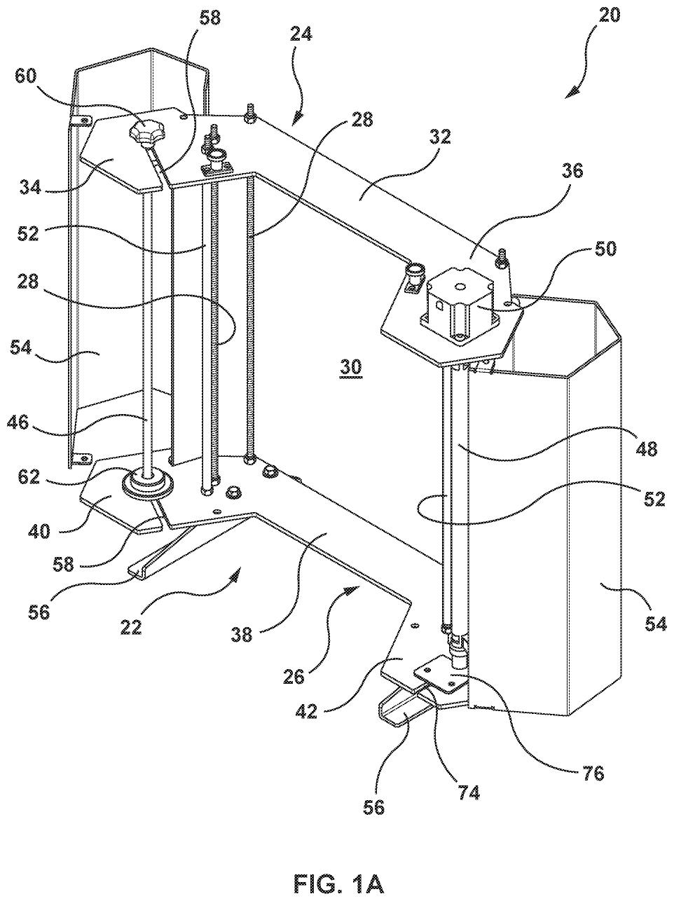

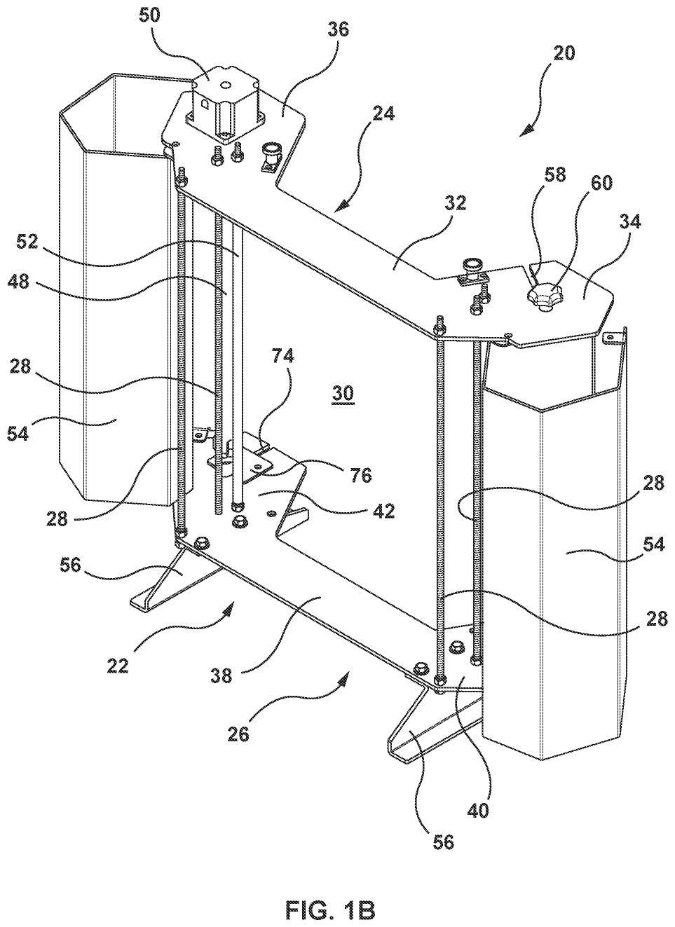

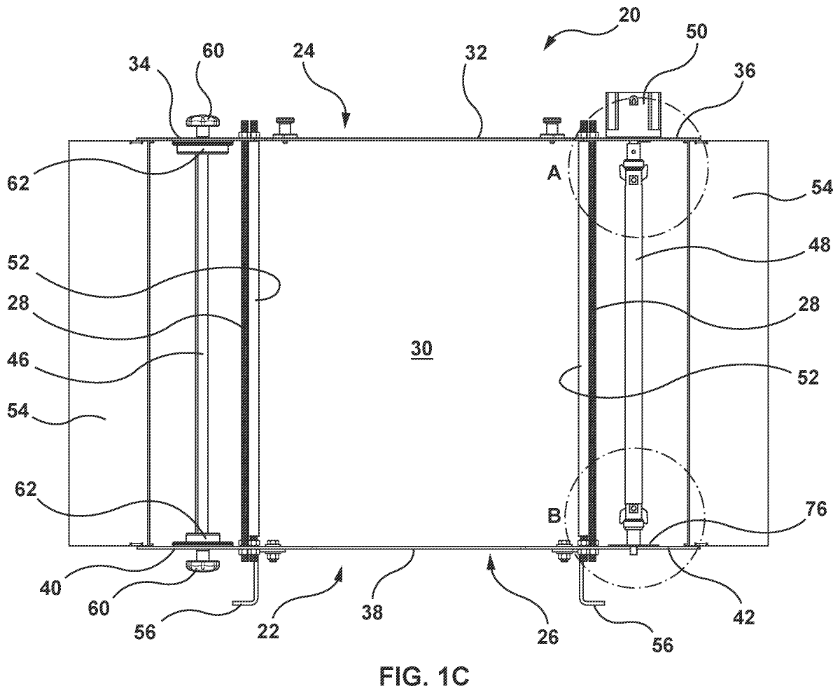

Referring initially to FIGS. 1A-C, there is shown a first embodiment of a protective barrier assembly 20. As illustrated, protective barrier assembly 20 includes a frame 22 that includes opposing top and bottom plates 24, 26 spaced apart by tie rods 28, which may in some cases be completely or partially threaded. Tie rods 28 are secured to respective drill holes formed in top and bottom plates 24, 26 by suitable fasteners such as screws, nuts, bolts, and the like. A total of four tie rods 28 are shown in FIGS. 1A-C, but additional (or fewer) tie rods 28 may be included in other embodiments of frame 22 depending on, e.g., size and structural considerations.

Top and bottom plates 24, 26 may in some embodiments have similar, although not necessarily identical, shapes and dimensionalities. For example, top plate 24 may include central leg 32 situated between lateral extensions 34,36 that project outward from central leg 32 at an angle therefrom. Likewise, bottom plate 26 may include a central leg 38 situated between lateral extensions 40,42 extending outwardly therefrom at an angle. The amount of angular deviation can vary in different configurations of top and bottom plates 24, 26 and may be zero in some embodiments, although it may be convenient in other cases to include an angular deviation, as described further below.

The spacing of top and bottom plates 24, 26 by tie rods 28 combined with the length of central legs 32, 38 defines a window 30. The size of the window 30 can be varied depending on the selected dimensionalities of these various components of frame 22. For example, as described herein below in greater detail, frame 22 may be formed so that window 30 corresponds approximately in size and shape to a user interface that is included as part of a computer terminal (e.g. of a kiosk, automatic banking machine (ABM), store checkout counter, etc.).

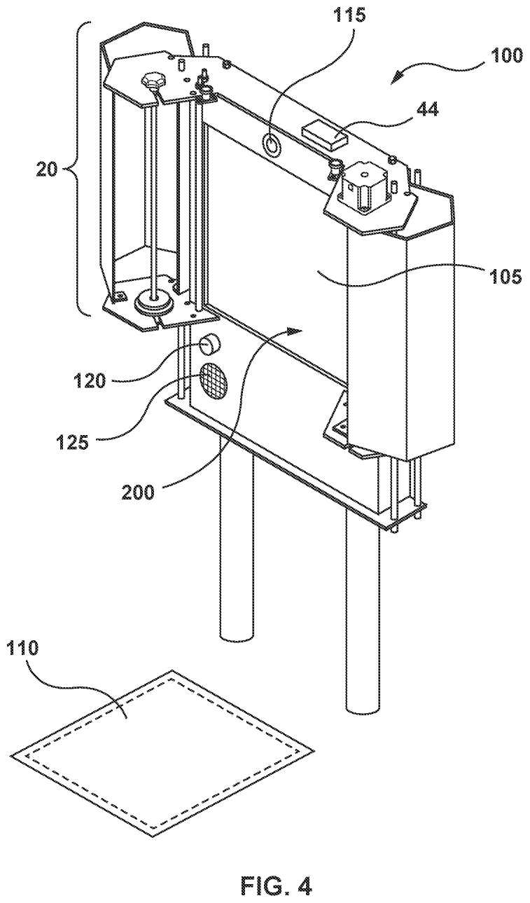

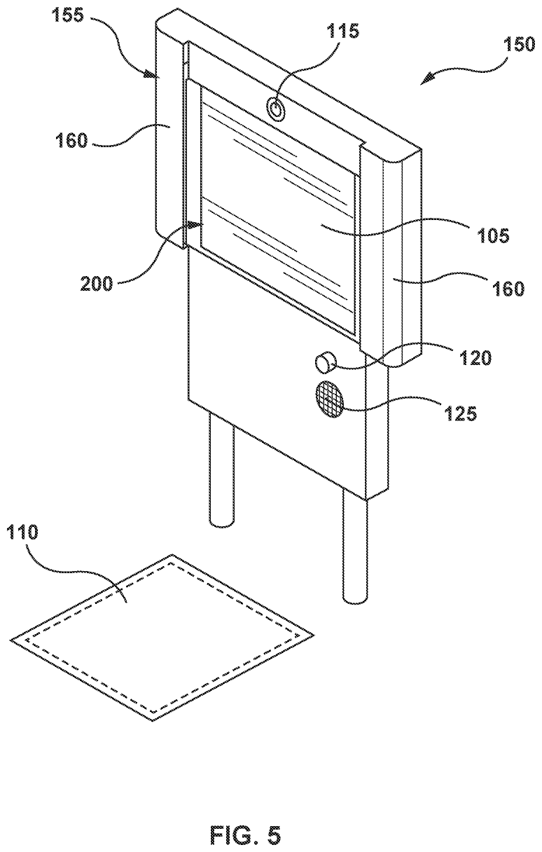

An axle 46 may be mounted to frame 22 on one side of window 30, for example, between lateral extensions 34, 40 of top and bottom plates 24, 26. Axle 46 may be fully or partially detachable from frame 22 and, as described further below with reference to FIGS. 4 and 5, carries rolls of a flexible protective barrier (not shown in FIGS. 1A to 1C), such as a film of linear low density polyethylene, on a feed reel (not shown in FIGS. 1A to 1C) that can be secured in place on axle 46. Detachment of axle 46 from frame 22 allows for loading and unloading of the feed reel from the assembly 20.

Mounted to frame 22 on the other side of window 30, generally opposite to axle 46, is a drive shaft 48 that anchors at one end to lateral extension 42 of bottom plate 26 and is operatively coupled at the other end to a motor 50 mounted in some cases to lateral extension 36 of top plate 32. Motor 50 can be any type of motor, such as a stepper motor or a DC motor paired with an encoder, timer, or other device which allows for control of the motor shaft's rotational position. Drive shaft 48 can be loaded with a collection reel (not shown in FIGS. 1A to 1C), so that rotation of motor 50 drives rotation of drive shaft 48, which in turn drives rotation of the collection reel.

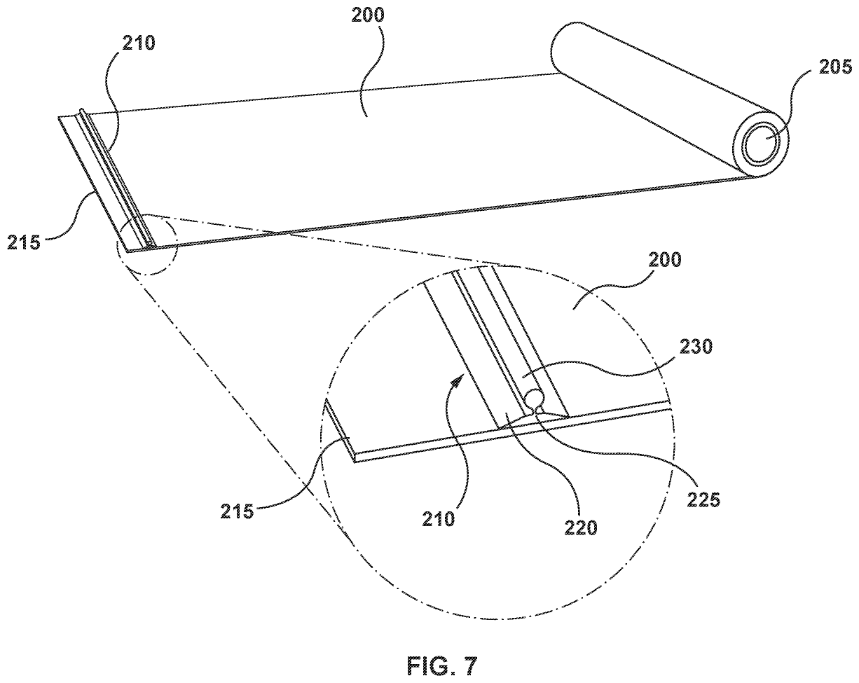

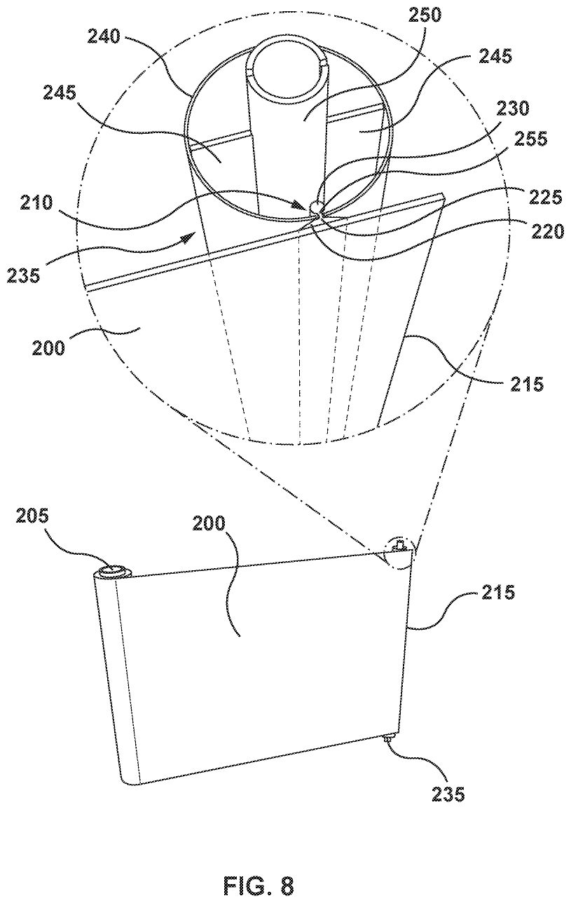

In use, a free end of the roll of protective barrier carried initially on the feed reel is securely attached to the collection reel (for example, using the connector described below with reference to FIGS. 7 and 8), so that the protective barrier is suspended and held taut between the feed reel and the collection reel, and so that a segment of the protective barrier is positioned in the window 30. By rotating the collection reel with motor 50, the protective barrier is advanced off of the feed reel and transferred onto the collection reel, so as to position fresh segments of the protective barrier in the window 30.

Like axle 46, drive shaft 48 may also be fully or partially detachable from frame 22 as well as from motor 50, although through a different mechanism to allow for operative coupling of drive shaft 48 to motor 50 when in use. The detachability of drive shaft 48 from frame 22 and/or motor 50 allows for loading of empty collection reels and, following transfer of the protective barrier from the feed reel, unloading of full collection reels.

As seen in FIG. 1C, rollers 52 are positioned on frame 22, for example, one on either side of window 30 in the general vicinity of axle 46 and drive shaft 48, respectively, supported between top and bottom plates 24,26. Rollers 52, which may generally take the form of any cylindrically shaped rotating rod or pole, re-direct the flexible protective barrier being advanced from the feed reel onto the collection reel so as to pass over or along window 30 in frame 22. Two rollers 52 are illustrated as an example, but in other embodiments of assembly 20, additional rollers 52 may be included, and the positioning of any or all rollers 52 may be varied, as needed or desired, to direct the protective barrier around objects or obstacles or otherwise alter the path of the protective barrier between the feed and collection reels.

Assembly 20 may also include a pair of housings 54, shown in the drawings as a hexagonal cylindrical enclosure of sufficient diameter to fit around the feed and collection reels when loaded onto frame 22. Housings 54 may be fully or partially detachable from frame 22 in order to provide access to the feed and collection reels, e.g. during loading and unloading. For example, in the example configuration shown, housings 54 include a hinge or other rotatable joint on which housings 54 may swivel combined with a clasp or other suitable enclosure used to secure housings 54 in place when closed. In some cases, housings 54 may also be completely removable from frame 22 or omitted completely.

Brackets 56 may also be mounted on frame 22 in some embodiments and used to attach assembly 20 externally to a computer terminal, kiosk, or the like that houses a touch-sensitive screen or other type of user interface. Brackets 56 may vary in size and shape to match the form factor of the computer terminal or kiosk to which assembly 20 is attached and are shown in the drawings as L-shaped legs for illustration only. In other embodiments, as seen in FIG. 4, for example, brackets 56 may be omitted and assembly 20 can in such cases be made to attach to the computer terminal or kiosk by clamping top and bottom plates 24,26 onto or around an external housing of the computer terminal or kiosk using tie rods 28.

Still with reference to FIGS. 1A and 1C, in one example configuration, axle 46 may be fitted into a pair of opposing slots 58 defined respectively in lateral extensions 34,40 of top and bottom plates 24,26. Knobs 60 or some other tightener hold axle 46 securely in place in slots 58. At any time, e.g., to access the feed reel, axle 46 can be detached from frame 22 by loosening knobs 60 and extracting axle 46 from slots 58. To re-attach axle 46 to frame 22, e.g., after being fitted with a new feed reel carrying a fresh roll of protective barrier, axle 46 can be re-positioned within slots 58 and secured in place by re-tightening knobs 60.

Optionally, feed reel spacers 62 may also be added to either end of axle 46 in order to assist with positioning and securement of the feed reel on axle 46. If included, feed reel spacers 62 can be used to axially clamp the feed reel in place on axle 46 when knobs 60 are tightened. Feed reel spacers 62 are depicted as bevelled cylindrical disks as an example and may be shaped differently in other configurations of assembly 20 or omitted altogether.

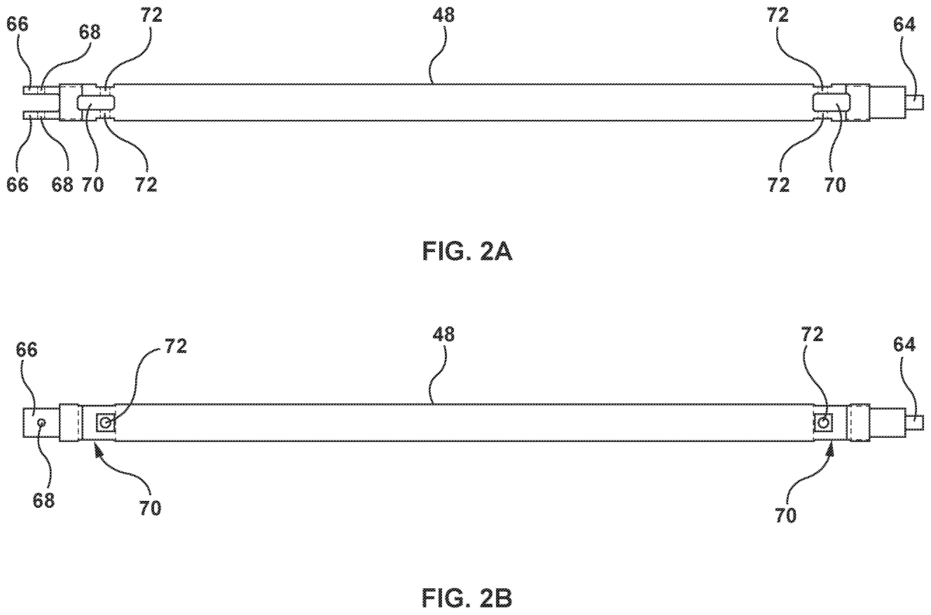

FIGS. 2A and 2B show an example embodiment of a drive shaft 48. As shown, drive shaft 48 may include a peg 64 extending axially at one end and a pair of opposing prongs 66 each formed with a pin hole 68 at the other end of drive shaft 48. Peg 64 may be generally cylindrical in shape, for example, and be sized to fit within a corresponding slot or drill hole in bottom plate 26 of frame 22. Prongs 66 are sized to mate securely with motor 50 in order to transfer torque generated by motor 50 onto drive shaft 48 and the collection reel secured thereon. In some embodiments, drive shaft 48 may also be formed with a pair of slots 70 positioned at either end, respectively, interiorly of peg 64 and prongs 66, and with orthogonal pin holes 72 cut therethrough.

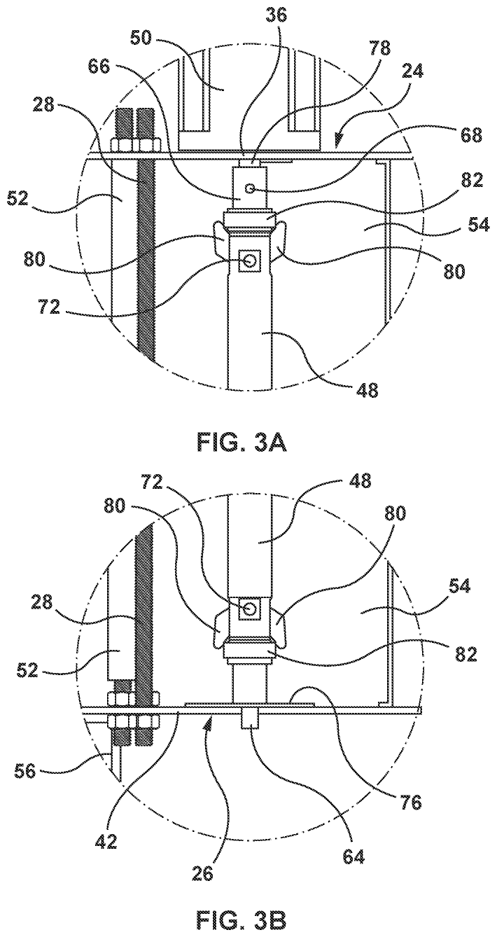

FIGS. 3A and 3B depict close up views of drive shaft 48 securely fastened to frame 22 and coupled to motor 50. The view in FIG. 3A corresponds to spotlight A in FIG. 1C and the view in FIG. 3B corresponds to spotlight B. In the example configuration shown, peg 64 of drive shaft 48 is grounded in a slot 74 formed in the lateral extension 42 of bottom plate 26 and secured in place by a drive shaft cover plate 76 fastened to bottom plate 26 (see also FIGS. 1A-1B) so as to restrain peg 64 within slot 74. In other embodiments, slot 74 and drive shaft cover plate 76 can be replaced with a through hole cut into bottom plate 26 into which peg 64 may be tightly inserted. In still other embodiments, peg 64 may be securely fastened to bottom plate 26, for example, using a ball-and-socket joint or other configuration of a hinge that allows drive shaft 48 to rotate about its anchor point on bottom plate 26. In such configurations, drive shaft 48 may be detachable from motor 50 but not from bottom plate 26. In other alternative configurations, drive shaft 48 may be anchored to a detachable cover fastened to bottom plate 26 around an opening through which drive shaft 48 may be extracted when the cover is detached.

At the other end of drive shaft 48, depicted in FIG. 3A, an output shaft 78 of motor 50 is projected through top plate 24 and received between prongs 66. For example, a through hole or other suitable opening may be cut into lateral extension 36 of top plate 24 into order to allow for coupling of output shaft 78 to prongs 66. A pin inserted through pin holes 68 and a corresponding pin hole cut into output shaft 78 may form a stable joint between output shaft 78 and drive shaft 48 through which to transfer torque when motor 50 is driven.

Removal of the pin from pin holes 68 allows for extraction of output shaft 78 from between prongs 66 and deconstruction of the joint. Drive shaft 48 may then be fully extracted from frame 22 by disengaging peg 64 from bottom plate 26, e.g., for the purpose of removing a fully spooled collection reel and replacing it with an empty one. The same procedure may then be followed in reverse order when remounting drive shaft 48 on frame 22 and coupling to motor 50. Alternatively, as described above, where drive shaft 48 is hinged to bottom plate 26, collection reels may be loaded and unloaded by uncoupling motor 50 and then rotating the free end of drive shaft 48 on its hinge out and away from frame 22. Alternatively, in some configurations, drive shaft 48 may be detached from frame 22 by de-coupling motor 50 and then extracting drive shaft 48 through an opening in bottom plate 26 as described above by detaching the plate cover.

Drive shaft 48 also in some configurations carries a pair of cams 80 in each slot 70 that are secured in place by, e.g., a pin inserted into pin holes 72 cut through each slot 70. Axial tightening of cam collars 82 threaded onto drive shaft 48 causes cams 80 to flare outwardly and grip a collection reel thereby transferring torsion and securing the collection reel in place on drive shaft 48. Cams 80 can likewise be disengaged from the collection reel by loosening of cam collars 82, e.g., in order to slide a fully spooled collection reel off of drive shaft 48 for removal.

One advantage to the use of cams 80 and cam collar 82 for securement of the collection reel is their relative positioning on drive shaft 48. For example, tighteners 60 and feed reel spacers 62 utilized in order to secure the feed reel in place on axle 46 may not be suitable for use in securing the collection feed on drive shaft 48, which unlike axle 46, operatively couples to motor 50. The combination of cams 80 and cam collar 82 can be positioned away from the end of drive shaft 48, which accommodates for the coupling of prongs 66 to output shaft 78. Thus, configurations of drive shaft 48 advantageously provide multiple functions simultaneously, namely, securement of the collection reel, operative coupling with motor 50, and detachability from the frame 22. Another advantage conferred by cams 80 and cam collars 82, described in further detail below, is that this combination allows for the removal of full collection reels making no or only incidental contact with the potentially contaminated roll of protective barrier that has been gathered thereon.

FIG. 4 depicts a computer terminal 100 that includes a touch-sensitive screen 105 in addition to other forms of user interface such as a button array, keypad, panel, and so on. Flexible barrier assembly 20 may generally attach to the external housing of computer terminal 100, so that the window 30 is aligned with touch-sensitive screen 105 (or at least a portion thereof that has been adapted to receive user input). In this position, flexible protective barrier 200 is suspended over the touch-sensitive screen 105, between users of computer terminal 100 and the touch-sensitive screen 105, which is thereby effectively shielded from user contact. Assembly 20 is depicted shielding a touch-sensitive screen 105 as an example only and in other cases may be deployed so as to shield a keypad, button array, or other type of user interface of a computer terminal 100 generally without limitation.

In the example configuration shown in FIG. 4, the top plate 24 of assembly 20 is engaged to the external housing of computer terminal 100 so that central leg 32 is flush with the top surface of computer terminal 100 and lateral extensions 34,36 extend outwardly to the side and forward. Likewise, lateral extensions 40,42 of bottom plate 26 project outwardly in a similar orientation and are fastened to lateral extensions 34,36 by way of tie rods 28 as described. Forward angling of the lateral extensions 34,36 and lateral extensions 40,42 conveniently positions the feed and collection rolls nearer to touch-sensitive screen 105, which facilitates positioning of the protective barrier 200 and can reduce the number of rollers 52 that are included in assembly 20. In this case, computer terminal 100 allows for central leg 38 of bottom plate 26 to be housed internally, but in other cases, bottom plate 26 will reside completely externally to computer terminal 100.

Being externally attachable to computer terminal 100, flexible barrier assembly 20 may function as a retrofit or after-market attachment for existing computer terminals of different shapes, sizes, and configurations. As noted above, the size and shape of window 30 is configurable by altering the dimensionalities of top and bottom plates 24,26 and tie rods 28, for example, such that window 30 may be made to fit a computer terminal 100 and touch-sensitive screen 105 of any shape or size generally without limitation. Additionally, as noted, the number and positioning of rollers 52 included in assembly 20, as well as the positioning of the feed and collection reels on frame 22, can be varied to alter the path of the protective barrier 200 and clear any obstacles or barriers that may be present.

When deployed on computer terminal 100, assembly 20 can first be enabled for use by loading a new or fresh roll of a flexible protective barrier 200, such as a film of linear low-density polyethylene. As described herein, the roll may be carried on a feed reel loaded onto axle 44 and fed around rollers 52 so as to overlay window 30 before being attached at a free end to an empty collection reel coupled to drive shaft 48 using, for example, the connector shown in FIGS. 7 and 8. The amount of tension experienced by the protective barrier 200 can be adjusted by winding and un-winding of the collection reel. In some cases, the collection reel can be wound until the protective barrier 200 is held taut between the feed and collection reels, but a degree of slack can also be introduced to the protective barrier by backing off or counter-rotation of the collection reel.

Protective barrier 200 may generally be constructed from a transparent and moisture impermeable material and can in some cases include one or more coatings, such as anti-fog, anti-glare, or anti-microbial preparations. Protective barrier 200 may additionally bear text, words, images, or other decorative and/or functional elements. For example, in some cases, protective barrier 200 can display instructional or marketing messages that would be visible to the user of computer terminal 100. In alternative embodiments, protective barrier 200 could be coated with time-sensitive inks that gradually fade over time, for example, to indicate when it is time to replace the protective barrier 200 with a new roll.

When drive shaft 48 is driven by motor 50, rotation of the collection reel pulls the protective barrier 200 to cause rotation of the feed reel, to cause new segments of flexible protective barrier 200 to be advanced off of the feed reel and into window 30 where user interaction with the touch-sensitive screen 105 occurs. At the same time, segments of the flexible protective barrier 200 that have been occupying the window 30 are drawn onto the collection reel and gradually spooled into another roll. When the entire roll of protective barrier 200 has been transferred, the now empty feed reel may be extracted from axle 46 and replaced with a new feed reel carrying a fresh roll of protective barrier 200. The collection reel can also be replaced with a new empty reel or, in some configurations, the existing collection reel can be replaced on drive shaft 48 after the roll of protective barrier 200 has been extracted, for example, as described further below.

When a user is present at computer terminal 100, protective barrier 200 shields the touch-sensitive screen 105 from the user and prevents direct physical contact from being made. However, the flexible of nature of the protective barrier 200, unlike rigid bodies such as plexiglass, still permits the transmission of gestures and other commands to the touch-sensitive screen 105. When a given user interaction with the touch-sensitive screen 105 is over, a new untouched segment of protective barrier 200 may be positioned in window 30 for a future interaction with a different user. In this manner, no two users make contact with the same surface and each user contacts a clean, previously untouched surface. As described herein, avoidance of contact with a common surface may be effective in mitigating or preventing the spread or surface transfer of germs, microbes, and other infectious agents from person to person.

To control user interactions with touch-sensitive screen 105, in some cases, protective barrier assembly 20 may further include an integrated control system 44 that is coupled to at least motor 50 (connection not shown explicitly) in addition to one or more sensors or ancillary components that provide input signals for control system 44. For example, assembly 20 may incorporate an internal sensor that tracks the amount of protective barrier 200 unspooled from the feed reel in order to match the length of new segments advanced from the feed reel to the width of window 30, as well as to detect when the end of the roll has been or is about to be reached. For example, a light indicator on a display or a message on touch-sensitive screen 105 could signal that the end of the roll has been reached and that assembly 20 has been disabled until a replacement roll of protective barrier 200 has been installed. In some cases, an internal sensor could also monitor for potential compromises to the integrity of the protective barrier 200 such as by way of puncture or accidental cutting when in use.

In some embodiments, computer terminal 100 may also include one or more additional sensors that provide control system 44 with signals used in the controlled advancement of protective barrier 200 by assembly 20. As one example, a pressure sensor or pad 100 installed on the floor may detect that a user is standing in front of the computer terminal 100 or that a user who had been standing in front of computer terminal 100 has now departed. As another example, a motion sensor 115 or camera may be installed on or within the computer terminal 100 and used to detect the approach or departure of the user from the vicinity of computer terminal 100.

In each of the above non-limiting examples, control system 44 is provided with an indication either that a particular user interaction with computer terminal 100 has been completed or that a new user interaction is about to be initiated. In either case, control system 44 may generally respond with a determination to replace the existing segment of protective barrier 200 positioned within window 30 with a new, unused segment from the feed reel (whether or not the segment that had been occupying window 30 had in fact been touched or contaminated by a user of computer terminal 100).

In some embodiments, control system 44 may also be configured to receive manual input from the user on the computer terminal 100 requesting advancement of the protective barrier 200. Manual input from the user could in some cases be provided by a command entered into the touch-sensitive screen 105 or, alternatively, via a separate button or input 120 located elsewhere on the computer terminal 100. As another example, computer terminal 100 could in some embodiments also house a microphone 125 that receives voice inputs from the user requesting advancement of protective barrier 200 and, like input button 120, transmits corresponding control signals to control system 44 to cause advancement of the protective barrier 200.

In some embodiments, control system 44 may also be configured to detect that a particular transaction, such as a financial transaction on a bank machine or POS or credit card terminal, or other user interaction on touch-sensitive screen 105 has ended and respond by automatically advancing a new segment of the protective barrier 200 into window 30. For example, control system 44 could detect the input of an end command or sequence by the user on touch-sensitive screen 105 or utilize a countdown timer or interrupt routine to identify periods of sufficient inactivity on touch-sensitive screen 105 based upon which control system 44 could determine that a transaction or interaction has ended.

FIG. 5 illustrates a computer terminal 150 that may provide an alternative to the embodiment of computer terminal 100 and protective barrier assembly 20 shown in FIG. 4. Computer terminal 150 differs from computer terminal 100 in that protective barrier assembly is integrated therein—i.e. some or all of the components of a protective barrier assembly are housed internally within computer terminal 150 as an “all-in-one” machine rather than attached externally, e.g., as a retrofit.

For example, in some embodiments, computer terminal 150 may be fitted with external housing 155 whose shape accommodates a protective barrier assembly substantially as described herein, while also providing users with access to a touch-sensitive screen 105. As shown, external housing 155 is formed with a pair of side lobes 160 that may be useful in accommodating one of more components of the protective barrier assembly, such as the feed and collection reels. However, other designs of external housing 155 where side lobes 160 are omitted may be possible as well depending on, e.g., the overall size and dimensions of computer terminal 150. Side lobes 160 can also in some cases be angled forwardly.

The configuration and operation of computer terminal 150 may otherwise be the same or similar to that of computer terminal 100 outfitted externally with a protective barrier assembly 20 and, for the sake of clarity and brevity, will not be described further other than to note possible differences existing therebetween with respect to configuration and/or principle of operation.

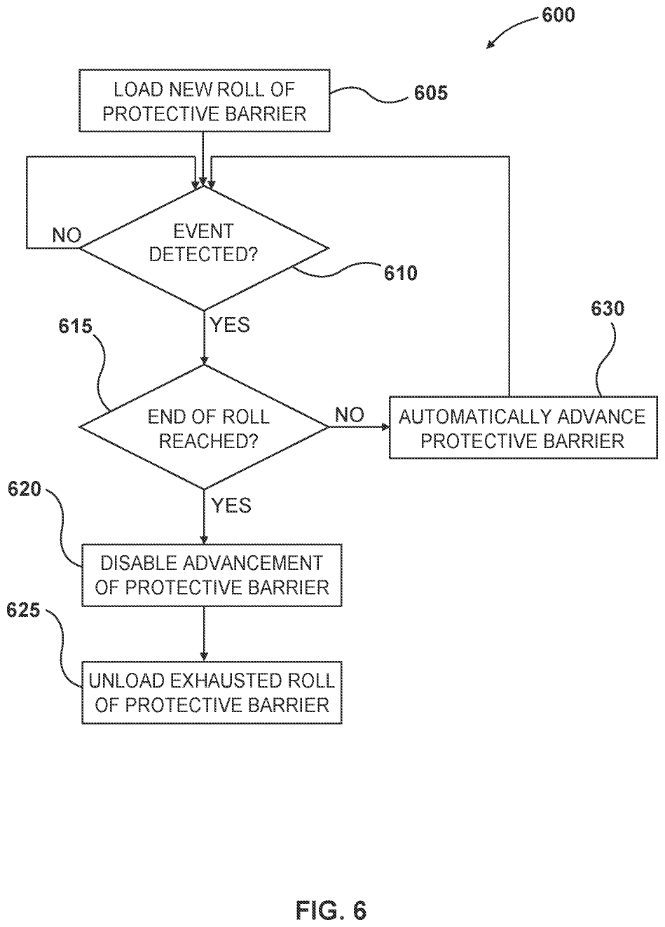

FIG. 6 illustrates a method 600 that may be used to control operation of a flexible barrier assembly as described herein. Method 600 may be performed on both a computer terminal 100 to which a flexible barrier assembly 20 has been externally attached as well as a computer terminal 150 that has been constructed with integrated components. Unless the contrary is expressly stated or implied by context, parts and sequences of method 600 as described may be altered, varied, performed in a different order, or omitted altogether according to different embodiments.

Accordingly, in some embodiments of method 600, at step 605 a roll of flexible protective barrier may be loaded onto a computer terminal and arranged to cover a touch-sensitive screen or other interface that is accessible to a user of the computer terminal. For example, the flexible protective barrier may be carried initially on a feed reel and attached at a free end to a collection reel as described herein and held therebetween in position and taut over the touch-sensitive screen.

While a computer terminal 100 or a computer terminal 150 is in operation, at step 610 periodic checks of one or more different sensors coupled to the computer terminal may be made by a control system to determine whether or not one or more events are detected at the computer terminal. As an alternative, events at the computer terminal may be registered by the sensors transmitting interrupt signals to the control system.

The event(s) detected by the one or more sensors may indicate to the control system the likelihood (although not necessarily the actuality) that a user interaction with a touch-sensitive screen 105 has either completed or is about to be initiated. For example, the event may be a pressure sensor or pad 110 installed on the floor detecting that a user is standing in front of a computer terminal or that a user who had been standing in front of computer terminal has now departed. Alternatively, the event may be a motion sensor 115 or camera installed on or within a computer terminal detecting either the approach or departure of the user from the vicinity of computer terminal.

Alternatively, the event may be the control system detecting or receiving notice of manual user input into either a touch-sensitive screen 105 or separate button 120 on the computer terminal requesting advancement of the protective barrier. Alternatively, the event may be a microphone 125 receiving a voice command from the user requesting advancement of the protective barrier.

Alternatively, the event may be the control system detecting or receiving notice that a particular sequence or financial or other transaction conducted on a touch-sensitive screen 105 by a user has concluded.

Following detection of the event by one or more sensors at step 610, at step 615 a determination may be made by the control system as to whether a protective barrier installed on the computer terminal has been completely or nearly completely unspooled, i.e., that there may be insufficient material remaining on the roll to advance a new segment that will completely cover the touch-sensitive screen and shield against user contact. If it is determined at step 615 that an end of the roll of protective barrier has been reached, then further advancement of new segments may be disabled by the control system at step 620, and the consumed roll of protective barrier may be unloaded from the computer terminal at step 625, e.g., to be replaced with a new unused roll of protective barrier that is loaded onto the computer terminal at step 605. For example, a service call requesting a replacement roll may be automatically placed or transmitted to a mobile device, etc. In some cases, at step 615, a determination may also be made whether or not the integrity of the protective barrier has been compromised and, if so, then further advancement of the protective barrier may again be disabled at step 620.

If it is not determined at step 615 that the end of the roll of protective barrier has been reached, then a new segment of protective barrier may be automatically advanced over the touch-sensitive screen 105 at step 630, and the control system can, at step 610, resume detection of future events that would result in automatic advancement of the protective barrier by the control system.

FIG. 7 shows an embodiment of a flexible protective barrier 200 spooled onto a feed reel 205 depicted, for example, as a hollow cylindrical core made out of cardboard, plastic, or some other suitably rigid material. As described herein, a feed reel 205 carrying protective barrier 200 may be loaded into the protective barrier assembly 20 of FIGS. 1A-C for use with a computer terminal 100, as well as loaded directly into a computer terminal 150, in either case to shield a touch-sensitive screen 105 or other form of interface from user contact.

In some embodiments, a connector 210 is applied to or near a free end 215 of protective barrier 200 that, as described herein, may be utilized to attach the free end 215 to a collection reel onto which protective barrier 200 may be gradually spooled during use. Connector 210 may take the form of a plastic extrusion or the like with a defined, e.g., uniform profile running the width of protective barrier 200 (or in some cases less than the entire width). For example, connector 210 may be shaped with a base layer or strip 220 that gradually tapers upwardly into a neck point 225 or other narrowing out of which a lobe 230 or other body is projected. Attachment of connector 210 to protective barrier 200 can be by way of welding, glue, adhesive strip, or any other suitable mechanical joint.

FIG. 8 shows protective barrier 200 attached at its free end 215 to an example embodiment of a collection reel 235 using connector 210. In this example configuration, collection reel 235 has a flexible outer shell 240 supported on one or more radial arms 245 of a rigid inner cylindrical core 250. A narrow slit or opening 255 in flexible outer shell 240 runs the length of collection reel 235. When connector 210 is pressed against slit 255, outer shell 240 deforms to an extent that allows lobe 230 of connector 210 to be fully received within slit 255 wherein lobe 230 is securely held by outer shell 240 rebounding back and pressing against neck 225 of connector 210 in which position outer shell 240 effectively restrains further movement of lobe 230.

To disengage connector 210 from the collection reel 235, the wings of outer shell 240 nestled against neck 225 may be retracted and deformed inwardly towards core 250 until lobe 230 has sufficient freedom and range of movement as to be manually extractable from slit 255. With connector 210 disengaged and outer shell 240 retained in a partially collapsed state, the entire roll of protective barrier 200 may be slipped off of the collection reel 235, or vice versa, at which point the outer shell 240 may be fitted with a new connector 210 carried on a different roll of protective barrier 200.

The configuration of collection reel 235 shown in FIG. 8 may conveniently facilitate disposal of a used protective barrier 200 that can reduce or completely avoid undue contact therewith. For example, once protective barrier 200 has been completely unrolled off of feed reel 205 and transferred onto the collection reel 235, a sealable bag or enclosure may be placed around the protective barrier 200 and the collection reel 235 then extracted as described above leaving behind the roll of protective barrier 200. The bag or enclosure containing the used protective barrier 200 may then be sealed off for disposal or recycling in some cases without coming into direct contact with the protective barrier 200 during the process.

In some alternative configurations, connector 210 may be omitted and the free end 215 of protective barrier 200 may be attached to collection reel 235 and held in place by wrap-fit engagement with outer shell 240. In such cases, the protective barrier 200 may be extracted, e.g., once completely transferred to the collection reel 235, as before, except for the lack of connector 210 to extract, by inwardly deforming the outer shell 240 until the collection reel 235 may again be slipped out leaving the collapsed roll of protective barrier 200 behind for disposal or recycling.

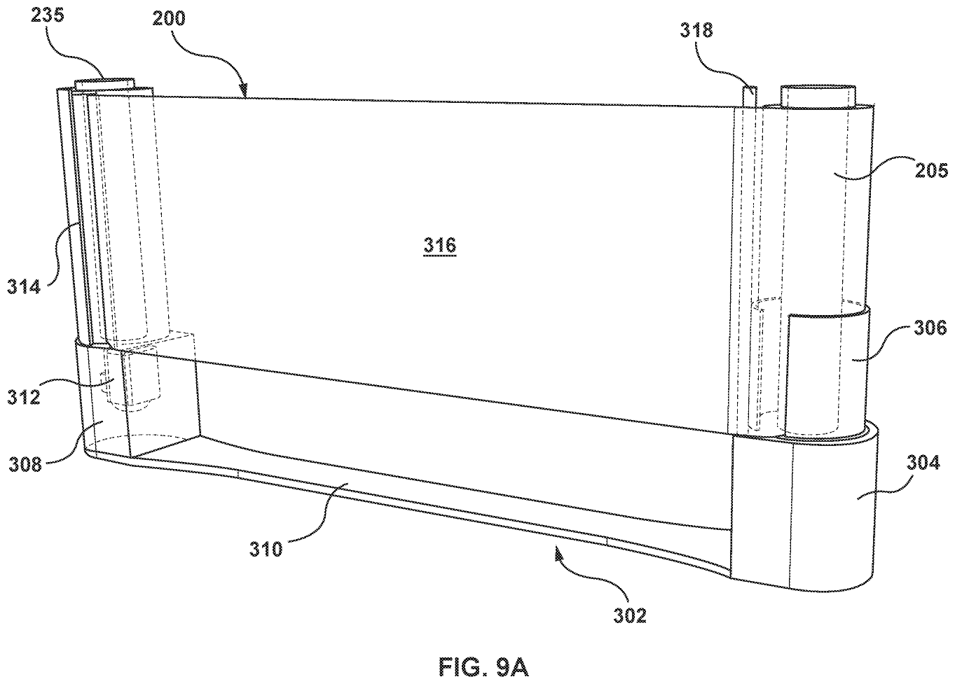

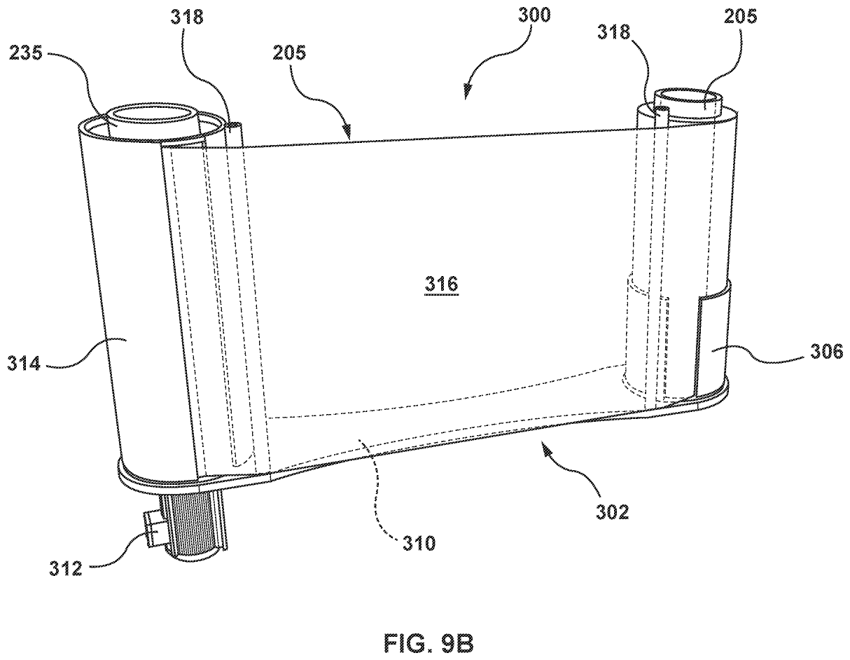

FIG. 9A illustrates a protective barrier assembly 300 that may provide an alternative to the embodiments of protective barrier assembly 20 shown in FIGS. 1A-C. Instead of being externally mountable on a computer terminal, protective barrier assembly 300 is suitable for installation or free-standing use on countertops, tables, desks, and other objects or surfaces where two more or individuals may interact with one another face to face in close proximity.

As is the case with common or shared surfaces, face to face interactions between people may also create an elevated risk of exposure to germs, microbes, bacteria, and other airborne pathogens. Protective barrier assembly 300 may be effective in reducing this risk by situating a segment of flexible protective barrier, as described herein, which acts as a physical barrier to stop airborne particles from travelling between two interacting or proximately located individuals.

As illustrated, protective barrier assembly 300 may include a frame 302 having a stand 304 that supports a feed reel 205 in a generally vertical or upright orientation. Feed reel 235 may carry a flexible protective barrier 200, such as a film of linear low-density polyethylene, which is substantially moisture impermeable and can be transparent. Collar 306 fixed to stand 304 may in some cases also be included in frame 302 to support and restrain feed reel 205 in its upright position but may also be omitted in other cases.

Motor housing 308 is located at an opposite end of base 302 rigidly coupled to stand 304 by bottom plate 310 that, together with base 304 and motor housing 308, provide a stable foundation for protective assembly 300 to stand upright free of others supports. Motor 312 is mounted within housing 308 and operatively coupled by way of a drive shaft to a collection reel 235 that, like the feed reel 205, is supported in a generally upright or vertical orientation and can in some cases be enclosed within a collection reel housing 314. Coupling of the collection reel 235 to the drive shaft may utilize a combination of cams and cam collars as described herein.

As with assembly 20 of FIGS. 1A-C, in use, a protective barrier 200 is unspooled from the feed reel 205 and can be attached at a free end to collection reel 235 using, for example, the connector 210 of FIGS. 7 and 8 (or in other cases by way of wrap-fit engagement). In this position, protective barrier 200 is suspended and held taut between the feed and collections reels 205,235 within a window 316 and between two or more people interacting with one another on either side of window 316. Motor 312 is used to control the advancement of new segments of protective barrier 200 into window 316 as described herein, for example, between successive user interactions across window 316. Rollers 318 mounted upright on stand 304 adjacent to the feed reel 205 and motor housing 308 adjacent to the collection reel 235 direct protective barrier 200 into window 316 and facilitate spooling and unspooling of protective barrier 200 during each advancement by motor 312.

In some cases, an air gap may be left under the protective barrier 200 and bottom plate 310 or the surface on which assembly 300 is installed or standing. However, the size of any air gap present can be reduced or eliminated if the base 302 is at least partially recessed within corresponding voids in the countertop or other surface on which assembly 300 is installed or standing.

FIG. 9B illustrates a variation of protective barrier 300 in which stand 304 and motor housing 308 are omitted and each of collar 306 and collection reel housing 314 are mounted directly on bottom plate 310. In contrast to the configuration shown in FIG. 9A, motor 312 is now exposed and positioned in relative terms outboard of or below bottom plate 310 (assuming an upright or vertical orientation of assembly 300).

Consequently, the example embodiment of protective barrier assembly 300 in FIG. 9B may be more suited to use on countertops or surfaces that have built-in recesses or chambers to accommodate motor 312. But, advantageously, elimination of stand 304 and motor housing 308 results in no or only a negligible air gap between the lower edge of protective barrier 200 and bottom plate 310 that rests essentially flush or level with the countertop or surface on which assembly 300 stands. This in turn can increase the overall effectiveness of barrier assembly 300 in stopping the transmission of airborne particles by covering, in relative turns, a larger percentage of window 316.

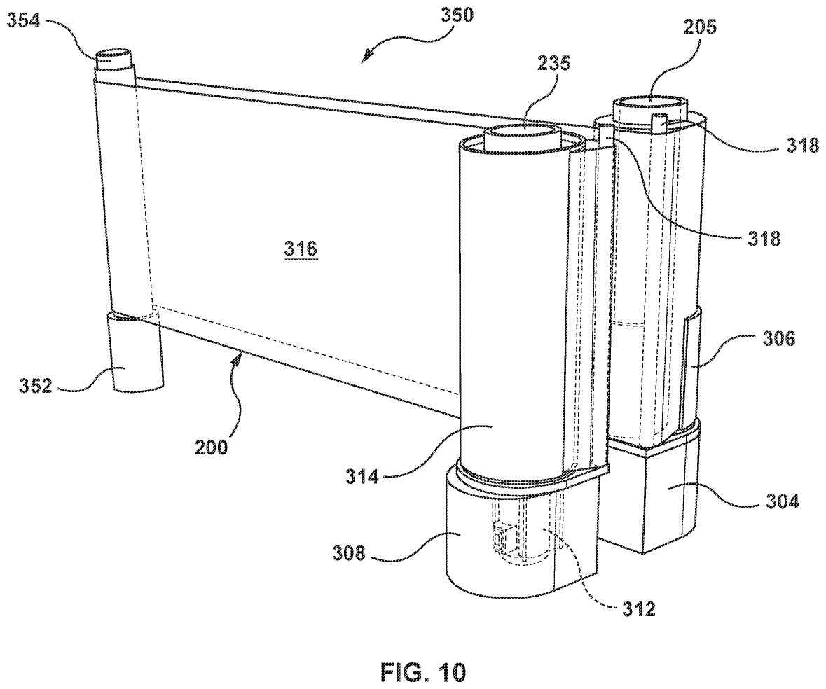

FIG. 10 illustrates a protective barrier assembly 350 that may provide another alternative embodiment in which a double layer of protective barrier is imposed in window 316 in place of a single layer. Protective barrier assembly 350 is similar to protective barrier assembly 300 in some aspects, but differs in that stand 304 is positioned adjacent to motor housing 308, bottom plate 310 is omitted, and a mooring 352 for an end roller 354 are included. For brevity and convenience, description of assembly 350 may be abbreviated.

As shown, the protective barrier 200 unspooled from feed reel 205 traverses window 316 to engage and wrap around end roller 354 before returning through window 316 in the opposite direction to be gathered onto collection reel 235 and to which it may be attached again at a free end using a connector 210, for example. In this manner, a double layer of protective barrier 200 is held in the window 316 between, on one side, the feed and collection reels 205,235, and end roller 354 on the other side of window 316.

With no bottom plate 310 included, it may be convenient to install protective barrier assembly 350 on countertops or surfaces with built in recesses or dock points for at least mooring 352, as well as stand 304 and motor housing 308 potentially, in order to maintain the structural stability of assembly 300 and to hold the protective barrier 200 taut between the feed and collection reels 205,235. However, other means of installation on a countertop or surface may be possible as well.

Protective barrier assembly 350, as too with assembly 300 in FIG. 9A and assembly 350 in FIG. 9B, is scalable in size based on the particular application. For example, protective barrier assembly 350 may in some cases be sized to fit on and cover a countertop, such as might be encountered in a store checkout counter, kiosk, or service window. However, in other cases, larger embodiments of protective barrier assembly 350 may be realized for use in physically separating, for example, tables in a restaurant or cubicles or workstations in an office building.

Use and operation of protective barrier assembly 350 may otherwise be as described herein with reference to other embodiments and will not be expanded further upon.

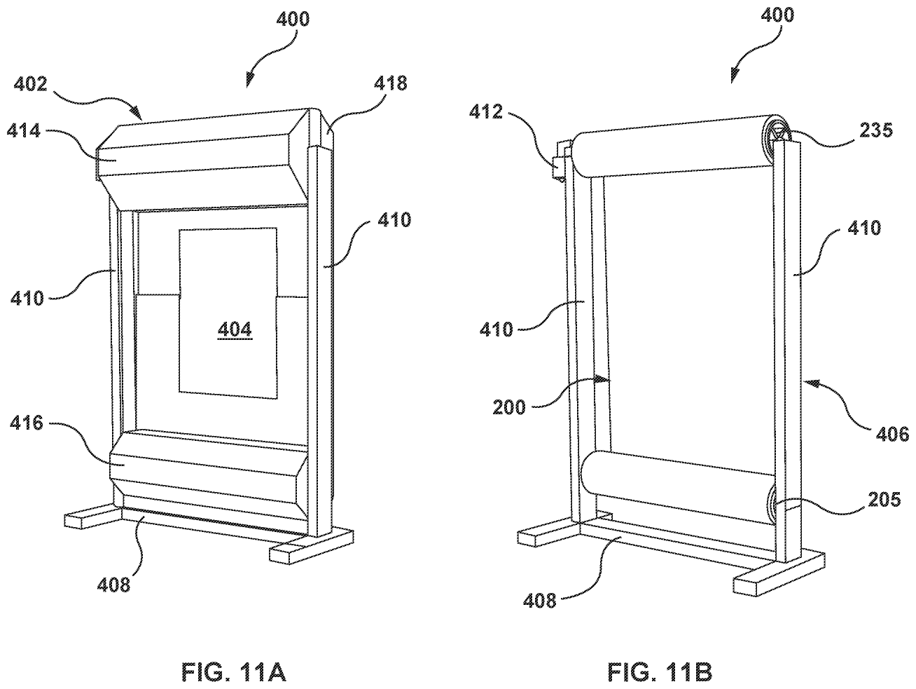

FIGS. 11A-B illustrate a protective barrier assembly 400 that may provide yet another alternative embodiment in which protective barrier 200 is suspended vertically between feed and collection reels 205,235 as opposed to horizontally. Protective barrier assembly 400 is shown in FIG. 11A fitted with an external housing 402 or enclosure that defines a window 404, and in FIG. 11B with the housing 402 removed to reveal feed and collection reels 205,235, as described herein, between which the protective barrier is suspended and held taut in position over window 404.

Protective barrier assembly 400 may include a frame 406 having a base 408 or stand on which are mounted a pair of opposed posts 410. Each of the feed reel 205 and collection reel 235 may be mounted to posts 410 horizontally, one above the other, at or near respective ends of the posts 410. In some cases, collection reel 235 may be mounted at the upper end of posts 410, operatively coupled to motor 412, with feed reel 205 mounted adjacent to base 408 (although the reverse is also possible in other configurations of assembly 400).

External housing 402 may be provided by one or more individual pieces and, in some cases, can include a collection reel housing 414, a feed reel housing 416, and a back sheet 418. In this way, access may be provided to either of the feed and collection reels 205, 235 individually by removing the corresponding piece of housing 402 as need be. However, housing 402 may also include additional or fewer pieces than what is shown in different embodiments.

Assembly 400 may in some cases be constructed with dimensions that are large enough that a person who is standing on one or the other side of assembly 400 can be effectively completely shielded from another person on the other side. For example, there may be many circumstances or situations in which a potentially contagious person is receiving medical treatment from a health care professional such as a doctor or nurse. Assembly 400 may be deployed in these and other situations to mitigate or lessen the risk of exposure to contagion by physically separating the patient receiving treatment and the health care professional.

The upright orientation and inclusion of window 404 in assembly 400 can be advantageous in multiple respects. For example, window 404 allows for a visual examination of the patient to be conducted while protective barrier 200, which is transparent, will effectively block the transmission of pathogens and other airborne particles. Additionally, by making a small slit or puncture in protective barrier 200, a health care professional may be able to directly administer a test to a patient, such as taking a temperature, collecting a sample using a cotton swab, or extracting a blood sample, while still remaining substantially behind protective barrier 200 and shielded from droplets or airborne particles released by the patient. Additionally, physical examination of the patient may also be possible with this configuration of assembly 400 by slackening or loosening the tension in the protective barrier 200, as described herein, to a degree which would allow adequate interaction between the health care professional and the patient.

Use and operation of protective barrier assembly 400 may otherwise be as described herein with reference to other embodiments and will not be expanded further upon.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A flexible barrier assembly comprising:
a frame defining a window;
feed and collection reels mounted on the frame;
a flexible protective barrier suspended and held taut between the feed and collection reels and overlaying the window; and
a motor operatively coupled to the collection reel to rotate the collection reel, wherein rotation of the collection reel by the motor causes advancement of the protective barrier from the feed reel to the collection reel.

2. The assembly of claim 1, wherein the frame is mountable to a computer terminal having a touch-sensitive screen accessible to a user through the window, and the protective barrier is positioned between the user and the touch-sensitive screen.

3. The assembly of claim 1, wherein the protective barrier comprises a connector at one end that is attachable to the collection reel.

4. The assembly of claim 3, wherein the collection reel comprises a flexible outer shell supported on a core, and the connector attaches to the collection reel though engagement with the outer shell.

5. The assembly of claim 4, wherein the collection reel is disengageable from the connector through inward deformation of the outer shell.

6. The assembly of claim 4, wherein the connector comprises an extruded lobe that is insertable into a linear opening formed in the outer shell wherein the extruded lobe is restrained.

7. The assembly of claim 1, wherein the amount of protective barrier advanced by the motor from the feed reel into the window is controllable.

8. The assembly of claim 7, further comprising a controller configured to operate the motor based on signals received from one or more sensors in communication with the controller.

9. The assembly of claim 8, wherein the controller instructs automatic advancement of the protective barrier by the motor in response to an event detected by the one or more sensors.

10. The assembly of claim 1, wherein the frame is free standing.

11. The assembly of claim 1, wherein the frame comprises a pair of rollers positioned one on either side of the window that orient the protective barrier over the window.

12. The assembly of claim 1, wherein the collection reel is mountable on a drive shaft that is detachably coupled to the motor and the frame.

13. The assembly of claim 12, wherein the drive shaft comprises one or more cams that secure the collection reel in place on the drive shaft.

14. The assembly of claim 12, wherein the drive shaft comprises a set of prongs at one end that releasably fasten to an output shaft of the motor.

15. A computer terminal comprising:
a touch-sensitive screen;
feed and collection reels mounted on either side of the touch-sensitive screen;
a flexible protective barrier that is suspended and held taut between the feed and collection reels and positioned to overlay the touch-sensitive screen;
a motor operatively coupled to the collection reel to rotate the collection reel, wherein rotation of the collection reel by the motor causes advancement of the protective barrier from the feed reel to the collection reel.

16. A method of controlling a flexible protective barrier that is suspended and held taut between feed and collection reels and positioned overlaying a window, the method comprising:
a. detecting an event; and
b. in response to detection of the event, automatically advancing the flexible protective barrier across the window by driving a motor.

17. The method of claim 16, wherein in response to detection of the event, a first segment of the protective barrier is removed from the window by the motor to be received onto the collection reel and a second segment of the protective barrier is advanced from the feed reel into the window.

18. The method of claim 16, wherein the event is detected by at least one sensor, and the event comprises at least one of the arrival of a user in the vicinity of a touch-sensitive screen or the departure of the user from the vicinity of the touch-sensitive screen.

19. The method of claim 16, wherein the event comprises receipt of manual input by a user on a touch-sensitive screen to advance the protective barrier.

20. The method of claim 16, where in the event comprises completion of a transaction by a user on a touch-sensitive screen.
